(19) United States
(12) Patent Application Publication
(10) Pub. No.: US 2003/0188753 A1
(43) Pub. Date: Oct. 9, 2003

(54) Radial angiography drape

(75) Inventor: Jerry T. Jascomb, Atlanta, GA (US)

Correspondence Address: KIMBERLY-CLARK WORLDWIDE, INC., 401 NORTH LAKE STREET, NEENAH, WI, 54956

(73) Assignee: Kimberly-Clark Worldwide, Inc.

(21) Appl. No.: 10/115,739

(22) Filed: Apr. 3, 2002

(51) Int. Cl. A61B 19/08

(52) U.S. Cl. 128/853

(57) ABSTRACT

A surgical drape for covering a patient during a surgical procedure is disclosed. The surgical drape includes a base sheet, a first fenestration formed in the base sheet through which a surgical procedure may be performed, an incise layer extending across the first fenestration, and an absorbent reinforcement panel disposed about the first fenestration. The reinforcement panel is positioned on the upper surface of the base sheet and has a reinforcement fenestration that is aligned with the first fenestration.

### BACKGROUND OF THE INVENTION

Drapes are used during surgical procedures to create and maintain a sterile environment around the surgical site. Draping materials are selected to create and maintain an effective barrier that minimizes the passage of microorganisms between non-sterile and sterile areas. To be effective, a barrier material should be resistant to blood, aqueous fluid, and abrasion, as lint-free as possible, and drapable. When used during surgery, drapes prevent blood and other bodily fluids from contaminating the sterile field.

A variety of surgical drapes exist, but most share several common features. Most drapes are made of a water-repellent or water-impermeable material, or are coated with such a material, to prevent the passage of bodily fluids as well as contaminating microorganisms. Many of today's surgical drapes are made of disposable nonwoven fabrics, plastic film, or papers.

Surgical drapes often have an opening or aperture (commonly known in the medical field as a “fenestration”) through which the surgical procedure is performed. In certain procedures, more than one surgical site is used. In these more complex procedures, the patient must be draped using a plurality of drapes or must be re-draped between procedures.

To minimize the costs associated with surgical procedures, there is currently a need for a one-piece drape that is easy to apply, provides multiple surgical sites, and may be tailored for specific types of surgery. Such an improved drape is provided by the present invention and will become more apparent upon further review of the following specification.

### SUMMARY OF THE INVENTION

The present invention relates to a surgical drape for covering a patient during a surgical procedure. The surgical drape includes a base sheet having an upper surface, a lower surface, a forward edge, and a rearward edge. The drape also includes a first fenestration formed in the base sheet through which a surgical procedure may be performed, the first fenestration formed in the base sheet such that when the drape is positioned on a patient, the first fenestration is disposed proximate to the radial artery in an arm of the patient. The drape may include an incise layer extending across the first fenestration, the incise layer having an adhesive side that is adapted to adhere to the patient, and an absorbent reinforcement panel disposed about the first fenestration, the reinforcement panel being positioned on the upper surface of the base sheet and having a reinforcement fenestration that is aligned with the first fenestration. The first fenestration may be disposed proximate to the radial artery in the right arm of the patient.

Another aspect of the present invention is a surgical drape having a base sheet with an upper surface, a lower surface, a forward edge, and a rearward edge, and a first fenestration formed in the base sheet such that when the drape is positioned on a patient, the first fenestration is disposed proximate to the radial artery in an arm of the patient. The first fenestration may be disposed proximate to the radial artery in the right arm of the patient. The drape may further include a second fenestration formed in the base sheet, an incise layer extending across the first and second fenestrations, and a reinforcement panel being positioned on the upper surface of the base sheet and having a reinforcement fenestration that is aligned with each of the first and second fenestrations. The second fenestration may be formed in the base sheet such that when the drape is positioned on a patient, the second fenestration is disposed proximate to the femoral artery of the patient.

Yet another aspect of the present invention includes a surgical drape having a base sheet with an upper surface, a lower surface, a forward edge, and a rearward edge, a first fenestration formed in the base sheet through which a surgical procedure may be performed, the first fenestration disposed proximate to the radial artery in the right arm of the patient, and a second fenestration formed in the base sheet proximate to the femoral artery in the right leg of the patient. The drape may include a third fenestration formed in the base sheet such that when the drape is positioned on the patient, the third fenestration is disposed proximate to the radial artery on the left arm of the patient, and a fourth fenestration formed in the base sheet such that when the drape is positioned on the patient, the fourth fenestration is disposed proximate to the femoral artery on the left leg of the patient. The drape may further include an incise layer extending across each of the first, second, third, and fourth fenestrations, the incise layer having an adhesive side that is adapted to adhere to the patient, and an absorbent reinforcement panel disposed about the first, second, third, and fourth fenestrations, the reinforcement panel being positioned on the upper surface of the base sheet and having a reinforcement fenestration that is aligned with each of the first, second, third, and fourth fenestrations.

### BRIEF DESCRIPTION OF THE DRAWINGS

FIG. 1 is a top view of a surgical drape;

FIG. 2 is a bottom view of a surgical drape;

FIG. 3 is a top view of a base sheet used in a surgical drape;

FIG. 4 is a top view of a reinforcement panel used with a surgical drape;

FIG. 5 is a bottom view of a reinforcement panel used with a surgical drape;

FIG. 6 is a broken away cross-sectional view of the surgical drape of FIG. 1 illustrating in part a release liner; and

FIG. 7 is a top view of a pouch used with a surgical drape.

### DESCRIPTION OF THE INVENTION

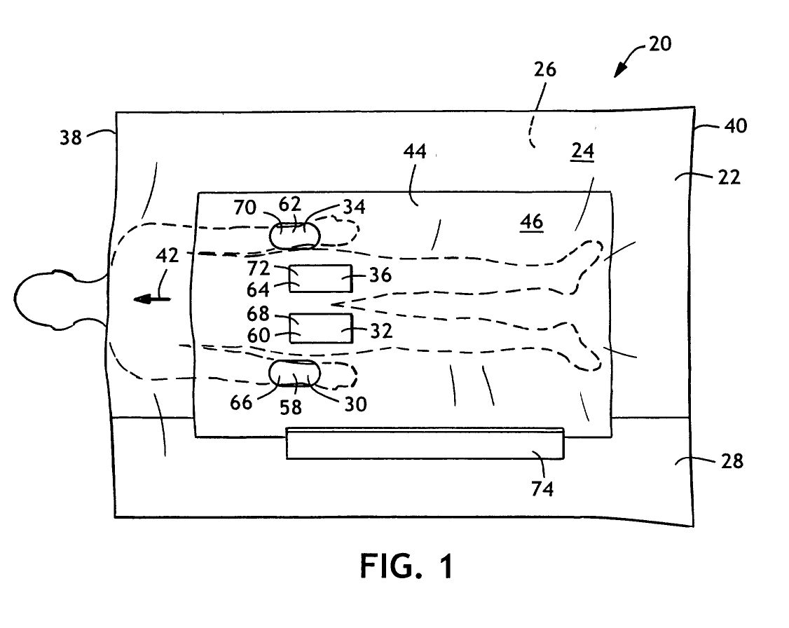

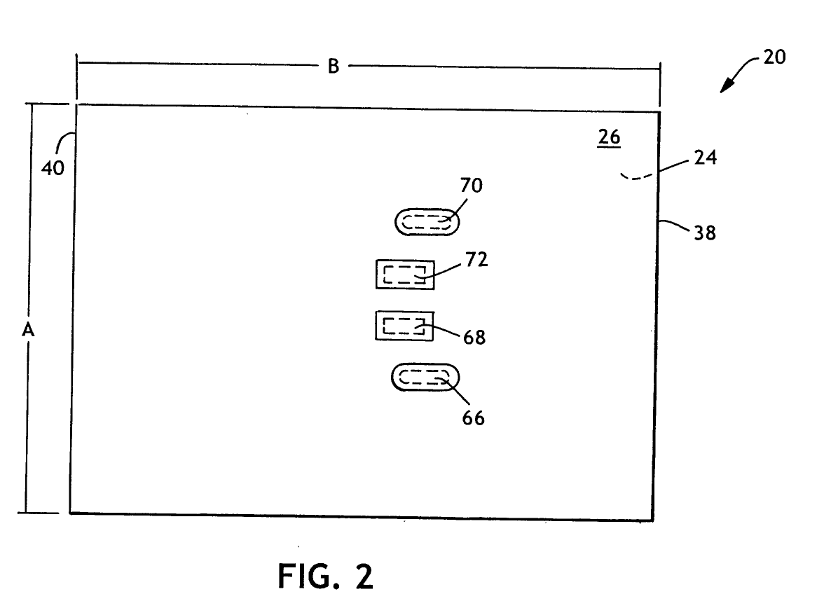

In response to the foregoing challenges that have been experienced by those of skill in the art, the present invention is directed toward a drape suitable for use in surgical procedures related to cardiology and angiography. The surgical drape 20 of the present invention is illustrated in FIG. 1 and includes a base sheet 22 having an upper surface 24 and a lower or patient-contacting surface 26, best shown in FIG. 2. Although it may have varying dimensions and shapes, the drape 20 is normally rectangular and sized to cover at least a majority of a patient's body during a surgical procedure.

The width A (FIG. 2) and the length B of the drape may vary. In selected embodiments, the width A may be within the ranges of 60 to 100 inches (152 to 254 cm), or 70 to 90 inches (178 to 229 cm), or 80 to 85 inches (203 to 216 cm). In some embodiments, the width A of the drape may be 82 inches (208 cm). The length B of the drape may also vary, but may be within the ranges of 80 to 160 inches (203 to 406 cm), or 110 to 150 inches (279 to 381 cm), or 120 to 140 inches (305 to 356 cm). In selected embodiments, the length B of the drape 20 may be 138 inches (350 cm). As used herein, any given range is intended to include all lesser included ranges. For example, a range of from 45-90 would also include the ranges of from 50-90, 45-80, 46-89, etc.

The base sheet 22 may be made from a wide variety of materials, including, for example, woven, reusable fabrics and nonwoven disposable fabrics or webs. Nonwoven materials suitable for use with the present invention include, for example, multilayer laminates such as a spunbond/meltblown/spunbond (“SMS”) material. An example of a suitable fabric is disclosed in U.S. Pat. No. 4,041,203, which is hereby incorporated by reference. The drape 20 (FIG. 1) may further include a viewing portion 28 made of a transparent, fluid resistant material, such as polyethylene film, for easy viewing and access to electrical table or c-arm controls and the like.

As used herein the term “nonwoven fabric or web” means a web having a structure of individual fibers or threads that are randomly interlaid, but not in an identifiable manner or pattern as in a knitted fabric. Nonwoven fabrics or webs have been formed from many processes such as for example, meltblowing processes, spunbonding processes, and bonded carded web processes. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91).

As used herein the term “spunbond fibers” or “spunbonded fibers” refers to small diameter fibers which are formed by extruding molten thermoplastic material as filaments from a plurality of fine, usually circular capillaries of a spinneret with the diameter of the extruded filaments then being rapidly reduced, for example, as in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al. Spunbond fibers are generally not tacky when they are deposited onto a collecting surface. Spunbond fibers are generally continuous and have average diameters (from a sample of at least 10) larger than 7 microns, more particularly, between about 10 and 20 microns.

As used herein the term “meltblown fibers” means fibers formed by extruding a molten thermoplastic material through a plurality of fine, usually circular, die capillaries as molten threads or filaments into converging high velocity, usually hot, gas (e.g. air) streams that attenuate the filaments of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin et al. Meltblown fibers are microfibers that may be continuous or discontinuous, are generally smaller than 10 microns in average diameter, and are generally tacky when deposited onto a collecting surface.

As used herein “multilayer laminate” means a laminate wherein some of the layers are spunbond and some meltblown such as a spunbond/meltblown/spunbond (SMS) laminate and others as disclosed in U.S. Pat. No. 4,041,203 to Brock et al., U.S. Pat. No. 5,169,706 to Collier, et al, U.S. Pat. No. 5,145,727 to Potts et al., U.S. Pat. No. 5,178,931 to Perkins et al. and U.S. Pat. No. 5,188,885 to Timmons et al. Such a laminate may be made by sequentially depositing onto a moving forming belt first a spunbond fabric layer, then a meltblown fabric layer and last another spunbond layer and then bonding the laminate in a manner described below. Alternatively, the fabric layers may be made individually, collected in rolls, and combined in a separate bonding step. Such fabrics usually have a basis weight of from about 0.1 to 12 osy (6 to 400 gsm), or more particularly from about 0.75 to about 3 osy. Multilayer laminates may also have various numbers of meltblown layers or multiple spunbond layers in many different configurations and may include other materials like films or coform materials, e.g. SMMS, SM, SFS, etc.

As used herein, the term “coform” means a process in which at least one meltblown diehead is arranged near a chute through which other materials are added to the web while it is forming. Such other materials may be pulp, superabsorbent particles, cellulose or staple fibers, for example. Coform processes are shown in commonly assigned U.S. Pat. Nos. 4,818,464 to Lau and 4,100,324 to Anderson et al. Webs produced by the coform process are generally referred to as coform materials.

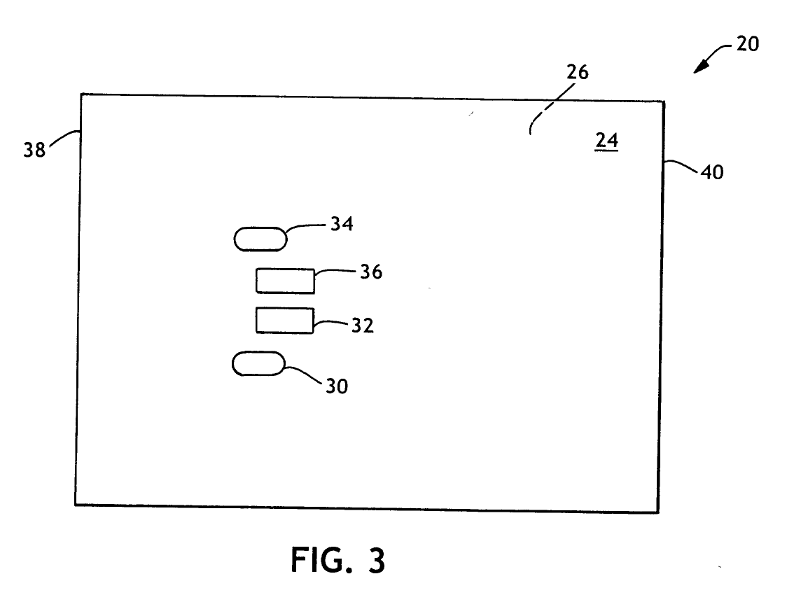

Referring now to FIG. 3, the base sheet 22 is shown therein and includes a first fenestration 30 and a second fenestration 32. The first fenestration 30 is oval in shape, while the second fenestration 32 is rectangular in shape. However, it is contemplated that any of the fenestrations utilized in the present invention may be of various shapes and sizes.

The first fenestration 30 is formed within the base sheet 22 so that when the drape 20 is applied to the patient, the first fenestration 30 is disposed over the surgical site for the cardiographic or angioplastic procedure. For example, if the patient is lying face up on the surgical table, the first fenestration 30 is disposed over at least a portion of the patient's arm so that the radial artery is available to the surgeon (FIG. 1). In some embodiments, the first fenestration 30 is disposed over at least a portion of the patient's right arm so that the radial artery in the right arm is available to the surgeon (FIG. 1). Where the electrical table, c-arm controls, and/or the like are provided on the right side of the table (as experienced by the patient lying face up on the table), use of the radial artery in the right arm affords the surgical personnel the ability to be conveniently proximal to both the artery on which the procedure is being performed and to such electrical and/or c-arm controls.

The second fenestration 32 is formed within the base sheet 22 so that when the drape 20 is applied to the patient, the second fenestration 32 is disposed over at least a portion of the patient's leg so that the femoral artery is available to the surgeon (FIG. 1). The femoral artery is made available as an alternate surgical site in the event that an attempt to perform the procedure using the radial artery is not successful or is otherwise infeasible. In some embodiments, the second fenestration 32 is disposed over at least a portion of the patient's right leg so that the femoral artery in the right leg is available to the surgeon (FIG. 1).

Additional fenestrations may be provided, such as a third fenestration 34 and a fourth fenestration 36, as shown in FIG. 1. The third fenestration 34 and fourth fenestration 36 provide a similar function as the first fenestration 30 and second fenestration 32, respectively. In some embodiments, the third fenestration 34 is positioned within the base sheet 22 so that, when the drape 20 is applied to the patient, the third fenestration 34 is disposed over at least a portion of the patient's arm so that the radial artery is available to the surgeon (FIG. 1). In some embodiments, the third fenestration 34 is disposed over at least a portion of the patient's left arm so that the radial artery in the left arm is available to the surgeon (FIG. 1). Likewise, the fourth fenestration 36 is positioned within the base sheet 22 so that, when the drape 20 is applied to the patient, the fourth fenestration 36 is disposed over at least a portion of the patient's leg so that the femoral artery is available to the surgeon (FIG. 1). In some embodiments, the fourth fenestration 36 is disposed over at least a portion of the patient's left leg so that the femoral artery in the left leg is available to the surgeon (FIG. 1). By providing multiple fenestrations in a single drape, the surgeon is afforded flexibility in performing the procedure using either the radial or femoral artery on either the right or left side of the patient without having to interrupt the procedure to replace the drape. This provides a significant advantage over drapes that do not anticipate such surgical complications and require additional drapes to perform the procedure.

The base sheet 22 further includes a forward edge 38 and a rearward edge 40 (FIG'S. 1, 2, and 3). The forward edge 38 is positioned toward the head of the patient to be covered, and the rearward edge 40 is positioned toward the feet of the patient to be covered (FIG. 1). Directions or other indicia may be stamped, printed or adhered to the drape to indicate how the drape is to be placed on the patient. For example, arrows such as arrow 42 (FIG. 1), as well as other diagrams may be utilized and applied to any portion of the surgical drape in any of a wide variety of manners.

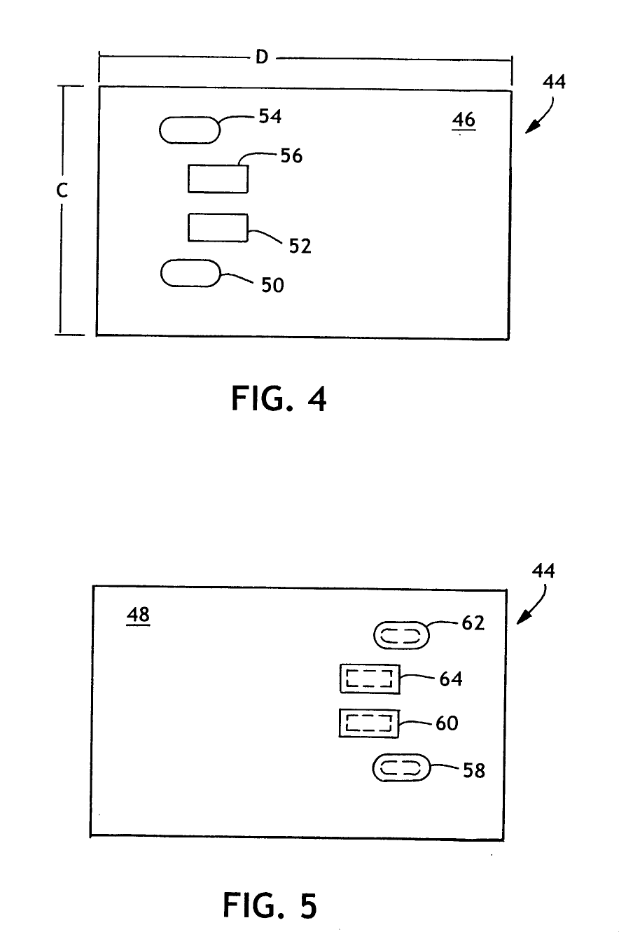

The surgical drape 20 of the present invention may further include a reinforcement panel 44 (FIG.'S 1, 4, and 5) having an upper surface 46 (FIG. 4) and a lower surface 48 (FIG. 5). As shown in FIG. 1, the reinforcement panel 44 is superimposed on and affixed in any suitable and appropriate manner to the upper surface 24 of base sheet 22. The width C (FIG. 4) and the length D of the panel may vary. In selected embodiments, the width C may be within the range of 30 to 40 inches (76 to 102 cm). In some embodiments, the width C of the panel 44 may be 35 inches (89 cm). The length D of the panel 44 may also vary, but may be within the range of 20 to 60 inches (51 to 152 cm). In selected embodiments, the length D of the panel 44 may be 48 inches (122 cm). As used herein, any given range is intended to include any and all lesser included ranges. For example, a range of from 45-90 would also include the ranges of from 50-90, 45-30, 46-89, etc.

The reinforcement panel 44 may be formed from a variety of materials, including a multilayer laminate which includes a fluid-absorbing material that may be backed by a fluid-repellent or fluid-impervious film layer. The film-layer side or lower surface 48 of the panel 44 is secured to the upper surface 24 of base sheet 22. A variety of attachment mechanisms may be used to secure the panel 44 to the upper surface 24 of the base sheet 22, for example, adhesive, stitching, thermal or ultrasonic bonding. The absorbent upper surface 46 of panel 44 remains exposed and available to absorb fluids emitted from the surgical site. The fluid-impervious film layer prevents the passage of blood and other body fluids through the reinforcement panel 44 and the base sheet 22. Although many commercially available materials are suitable for use in the reinforcement panel 44, an exemplary material is available from Kimberly-Clark Corporation and is marketed under the trade name NEW CONTROL PLUS®).

In some embodiments, the upper surface 46 of the reinforcement panel 44 may have an increased coefficient of friction to provide a slip-resistant surface to lessen the likelihood of undesired movement of surgical instruments that are placed upon the reinforcement panel 44. The coefficient of friction may be increased by providing a textured surface or by any other means known to those of skill in the art. The reinforcement panel 44 may be constructed of a material that has an absorbent upper surface to absorb fluids near the operative site. The reinforcement panel 44 also helps to inhibit penetration of the drape 20 by instruments that are placed on top of the reinforcement panel 44 during surgery.

As shown in FIG. 1, the reinforcement panel 44 may be disposed about the first fenestration 30, and in some embodiments, also about the second fenestration 32. In some embodiments, the reinforcement panel 44 is disposed about the third fenestration 34 (FIG. 1). In yet other embodiments, the reinforcement panel is disposed about the fourth fenestration 36 (FIG. 1). The reinforcement panel 44 may be positioned on the upper surface 24 of the base sheet 22 and may include reinforcement fenestrations that are aligned with the first fenestration 30, second fenestration 32, third fenestration 34, and/or fourth fenestration 36 (FIG. 4). As shown in FIG. 4, fenestrations 50, 52, 54, and 56 are formed in the panel 44 that, when the panel 44 is adhered to the base sheet 22, are aligned with the fenestrations 30, 32, 34, and 36 (FIG.'S 1 and 2) formed in the base sheet 22. Specifically, the fenestration 50 formed in the reinforcement panel 44 is aligned with the first fenestration 30 when the panel 44 is adhered to the base sheet 22. Similarly, the fenestration 52 formed in the reinforcement panel 44 is aligned with the second fenestration 32 when the panel 44 is adhered to the base sheet 22. Where additional fenestrations 34 and 36 are provided (FIG. 1), the reinforcement layer 40 may include corresponding fenestrations 54 and 56 (FIG. 4) that align with fenestrations 34 and 36 when the reinforcement panel 44 is adhered to the base sheet 22.

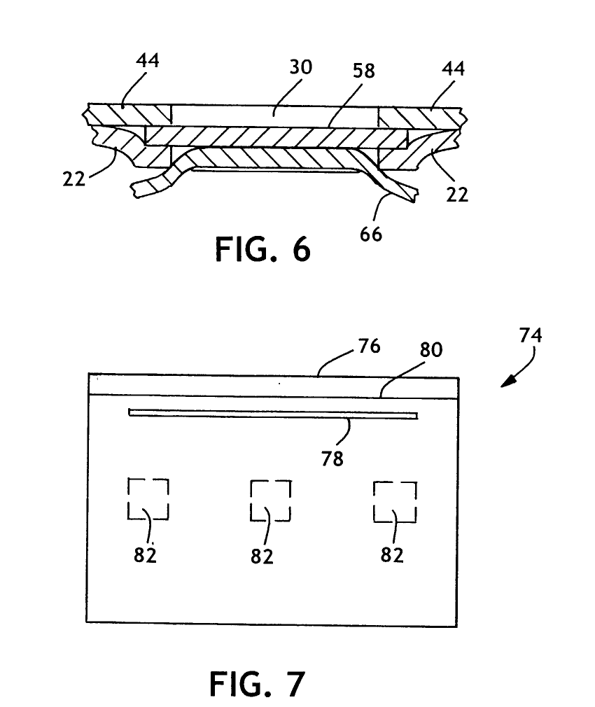

Referring now to FIG. 5, an incise layer 58 may be provided and positioned over the first fenestration 30 (FIG. 1). The incise layer 58 may be formed from a low-density polyethylene film with adhesive on one side. For example, the incise layers may be constructed from polyethylene film available from Bertek Inc., St. Albans, Vt. 05478, or from a film available from Medical Concepts Development, Inc., St. Paul, Minn. 55125. In some embodiments, the incise layers may be constructed from an adhesive film available from 3M, Minneapolis, Minn. under the trade name 1525L. The incise layer 58 is, in the embodiment depicted in FIG. 1, disposed between the reinforcement panel 44 and the base sheet 22. As shown in FIG. 6, the incise layer 58 is secured to the lower surface 48 of the panel 44. If desired, an incise layer 60 may be applied over the second fenestration 32 (FIG. 5). The incise layers 58 and 60 may include an adhesive side that is adapted to adhere to the patient when the drape 20 is covering the patient. Thus, the adhesive side of the incise layer 58 or 60 faces downwardly when the drape 20 is positioned over the patient to provide a seal around the surgical site. Where a third fenestration 34 or a fourth fenestration 36 is provided, corresponding incise layers 62 and/or 64 may be included. In some embodiments, the incise layer may include a fenestration that may correspond in shape to any of fenestrations 30, 32, 34 and/or 36, where provided. However, it is contemplated that a fenestration provided in the incise layer may be of any shape or size.

In other embodiments, strips of adhesive (not shown) may be positioned around the periphery of the fenestrations 30, 32, 34, and 36 (FIG. 1) to adhere the periphery of the fenestrations to the patient. The tacky and pressure-sensitive adhesives used may be of any biologically acceptable adhesive. Examples of such adhesive materials are described in U.S. Pat. No. 3,669,106 entitled “Surgical Drape with Adhesive Attachment Means” to Schrading et al., which is incorporated herein in its entirety by reference.

To facilitate handling of the drape 20 and to maintain the sterility of the incise layers 58 and 60 or, in selected embodiments, the peripheral adhesive strips (not shown), the adhesive surface may be covered with a release liner such as, for example, release liners 66 and 68 shown in FIG. 2. The release liners may be formed of any of a wide variety of materials which are commonly available. For example, wax- or silicone-coated papers may be placed over the adhesive side of the incise layers until the drape 20 is applied to the patient. Alternate materials may also be utilized, such as, for example, plastic materials having at least one non-adherent surface. Such materials may be utilized when a tear-resistant release liner is appropriate. Additionally, the release liners 66 and 68 may be segmented to facilitate application of the drape 20 to the patient (not shown). Where a third fenestration 34 or a fourth fenestration 36 is provided with corresponding incise layers 62 and 64, release liners 70 and/or 72 may be included.

As shown in FIG. 1, at least one pouch 74 (FIG. 7) may be attached to the surgical drape 20. The pouch 74 may be utilized to hold surgical instruments, collect fluids or a variety of other similar functions. The pouch 74 may be adhered to the reinforcement panel 44 or to the base sheet 22 by adhesive such as tape and the like, stitching, thermal bonding, adhesive bonding, ultrasonic bonding, or other commonly known attachment mechanisms. In some embodiments, the top edge 66 of the pouch is adhered to the upper surface 24 of the reinforcement panel 44 (FIG. 1). The pouch 74 may include a malleable wire or strip 78 that may be positioned along the outside portion of the pouch 74 beneath the opening 80 of the pouch 74 to maintain the pouch 74 in an open position (FIG. 7).

The lower portion of the pouch 74 may be releasably secured to the upper surface 24 of the base sheet 22 by hook-type fasteners such as fasteners 82 (FIG. 7). Such hook and loop fasteners 82 are well known, and the use of such hook-type fasteners 82 with nonwoven materials such as surgical drapes is common. The fasteners 82 are applied to the rear surface of the pouch 74 that will come into contact with the upper surface 24 of the base sheet 22. The use of fasteners 82 enables the pouch 74 be utilized to retain electric cords, suction lines and the like which commonly run alongside the patient. To secure such cords and lines, the lower surface of the pouch 74 is moved away from the upper surface 24 of the base sheet 22, and the cords and lines are positioned (not shown) between the under side of the pouch 74 and the upper surface of the base sheet 22. The lines and cords are routed above the fasteners 82, which are then secured to the upper surface 24 of the base sheet 22. Thus, the cords and lines are retained securely between the pouch 74 and base sheet 22.

In some embodiments, two or more pouches may be provided (not shown), each being positioned adjacent to a fenestration 30, 32, 34, or 36. The pouch 74 is, in some embodiments, formed of a material that is impervious to liquids, such as, for example, polyethylene or the like. The pouch 74 may be formed of a transparent or opaque material.

The drape 20 may be folded in a variety of manners to assist the medical personnel in applying the drape 20 to the patient. The surgical drape 20 of the present invention may be folded so that, as medical personnel apply the drape to the patient, the lower surface 26 of the drape 20 is on the exterior of the folded drape (not shown). The release liners 66, 68, 70, and 72 covering fenestrations 30, 32, 34, and 36 respectively, are clearly visible and easily accessible by medical personnel. In embodiments where the release liner 66 that is disposed over the incise layer 58 of the first fenestration 30 is segmented, the medical personnel applying the drape 20 to the patient may expose only a portion of the adhesive side of the incise layer 58 (not shown). This facilitates application of the drape 20 to the patient while minimizing the opportunities for contamination of the incise layer 58. While the invention has been described in detail with respect to specific preferred embodiments thereof, it will be appreciated that those skilled in the art, upon attaining an understanding of the foregoing, may readily conceive of alterations to and variations of the preferred embodiments. Such alterations and variations are believed to fall within the scope and spirit of the invention and the appended claims.

What is claimed is:

1. A surgical drape comprising:
a base sheet having an upper surface, a lower surface, a forward edge, and a rearward edge;
a first fenestration formed in the base sheet such that when the drape is positioned on a patient, the first fenestration is disposed proximate to the radial artery in an arm of the patient;
an incise layer extending across the first fenestration and having an adhesive side that is adapted to adhere to the patient; and
an absorbent reinforcement panel disposed about the first fenestration, the reinforcement panel being positioned on the upper surface of the base sheet and having a reinforcement fenestration that is aligned with the first fenestration.

2. The drape of claim 1, wherein the first fenestration is disposed proximate to the radial artery in the right arm of the patient.

3. The drape of claim 1, further comprising a second fenestration formed in the base sheet through which a surgical procedure may be performed.

4. The drape of claim 3, wherein the second fenestration is formed in the base sheet such that when the drape is positioned on a patient, the second fenestration is disposed proximate to the femoral artery of the patient.

5. The drape of claim 4, wherein the second fenestration is disposed proximate to the femoral artery in the right leg of the patient.

6. The drape of claim 3, further comprising a third fenestration formed in the base sheet such that when the drape is positioned on the patient, the third fenestration is disposed proximate to the radial artery on the left arm of the patient.

7. The drape of claim 6, further comprising a fourth fenestration formed in the base sheet such that when the drape is positioned on the patient, the fourth fenestration is disposed proximate to the femoral artery on the left leg of the patient.

8. The drape of claim 3, wherein the reinforcement panel is disposed about the first and second fenestrations.

9. The drape of claim 1, further comprising at least one pouch attached to the drape.

10. A surgical drape comprising:
a base sheet having an upper surface, a lower surface, a forward edge, and a rearward edge;
a first fenestration formed in the base sheet such that when the drape is positioned on a patient, the first fenestration is disposed proximate to the radial artery in an arm of the patient;
a second fenestration formed in the base sheet; an incise layer extending across the first and second fenestrations, the incise layer having an adhesive side that is adapted to adhere to the patient; and
an absorbent reinforcement panel disposed about the first and second fenestrations, the reinforcement panel being positioned on the upper surface of the base sheet and having a reinforcement fenestration that is aligned with each of the first and second fenestrations.

11. The drape of claim 10 wherein the first fenestration is formed in the base sheet so that it is proximate to the radial artery in the right arm of the patient.

12. The drape of claim 10, wherein the second fenestration is formed in the base sheet such that when the drape is positioned on a patient, the second fenestration is disposed proximate to the femoral artery of the patient.

13. The drape of claim 12, wherein the second fenestration is disposed proximate to the femoral artery in the right leg of the patient.

14. The drape of claim 11, further comprising a third fenestration formed in the base sheet such that when the drape is positioned on the patient, the third fenestration is disposed proximate to the radial artery on the left arm of the patient.

15. The drape of claim 14, further comprising a fourth fenestration formed in the base sheet such that when the drape is positioned on the patient, the fourth fenestration is disposed proximate to the femoral artery on the left leg of the patient.

16. The drape of claim 10, further comprising at least one pouch attached to the drape.

17. A surgical drape comprising:
a base sheet having an upper surface, a lower surface, a forward edge, and a rearward edge;
a first fenestration formed in the base sheet through which a surgical procedure may be performed, the first fenestration formed in the base sheet such that when the drape is positioned on a patient, the first fenestration is disposed proximate to the radial artery in an arm of the patient;
a second fenestration formed in the base sheet through which a surgical procedure may be performed, the second fenestration formed in the base sheet such that when the drape is positioned on a patient, the second fenestration is disposed proximate to the femoral artery of the patient;
an incise layer extending across the first and second fenestrations, the incise layer having an adhesive side that is adapted to adhere to the patient; and
an absorbent reinforcement panel disposed about the first and second fenestrations, the reinforcement panel being positioned on the upper surface of the base sheet and having a reinforcement fenestration that is aligned with each of the first and second fenestrations.

18. The drape of claim 17 wherein the first fenestration is disposed proximate to the radial artery in the right arm of the patient.

19. The drape of claim 18, further comprising a third fenestration formed in the base sheet through which a surgical procedure may be performed, the third fenestration formed in the base sheet such that when the drape is positioned on the patient, the third fenestration is disposed proximate to the radial artery on the left arm of the patient.

20. The drape of claim 19, wherein the second fenestration is disposed proximate to the femoral artery in the right leg of the patient.

21. The drape of claim 20, further comprising a fourth fenestration formed in the base sheet through which a surgical procedure may be performed, the fourth fenestration formed in the base sheet such that when the drape is positioned on the patient, the fourth fenestration is disposed proximate to the femoral artery on the left leg of the patient.

22. The drape of claim 17, further comprising at least one pouch attached to the drape.

23. A surgical drape comprising:
a base sheet having an upper surface, a lower surface, a forward edge, and a rearward edge;
a first fenestration formed in the base sheet through which a surgical procedure may be performed, the first fenestration formed in the base sheet such that when the drape is positioned on a patient, the first fenestration is disposed proximate to the radial artery in the right arm of the patient;
a second fenestration formed in the base sheet through which a surgical procedure may be performed, the second fenestration formed in the base sheet such that when the drape is positioned on a patient, the second fenestration is disposed proximate to the femoral artery of the patient;
a third fenestration formed in the base sheet through which a surgical procedure may be performed, the third fenestration formed in the base sheet such that when the drape is positioned on the patient, the third fenestration is disposed proximate to the radial artery on the left arm of the patient;
an incise layer extending across the first, second, and third fenestrations, the incise layer having an adhesive side that is adapted to adhere to the patient; and
an absorbent reinforcement panel disposed about the first, second, and third fenestrations, the reinforcement panel being positioned on the upper surface of the base sheet and having a reinforcement fenestration that is aligned with each of the first, second, and third fenestrations.

24. The drape of claim 23, further comprising a fourth fenestration formed in the base sheet through which a surgical procedure may be performed, the fourth fenestration formed in the base sheet such that when the drape is positioned on the patient, the fourth fenestration is disposed proximate to the femoral artery on the left leg of the patient.

25. The drape of claim 23, further comprising at least a first pouch attached to the drape.

26. The drape of claim 25, further comprising at least a second pouch attached to the drape.

27. A surgical drape comprising:
a base sheet having an upper surface, a lower surface, a forward edge, and a rearward edge;
a first fenestration formed in the base sheet through which a surgical procedure may be performed, the first fenestration formed in the base sheet such that when the drape is positioned on a patient, the first fenestration is disposed proximate to the radial artery in the right arm of the patient;
a second fenestration formed in the base sheet through which a surgical procedure may be performed, the second fenestration formed in the base sheet such that when the drape is positioned on a patient, the second fenestration is disposed proximate to the femoral artery in the right leg of the patient;
a third fenestration formed in the base sheet through which a surgical procedure may be performed, the third fenestration formed in the base sheet such that when the drape is positioned on the patient, the third fenestration is disposed proximate to the radial artery on the left arm of the patient;
a fourth fenestration formed in the base sheet through which a surgical procedure may be performed, the fourth fenestration formed in the base sheet such that when the drape is positioned on the patient, the fourth fenestration is disposed proximate to the femoral artery on the left leg of the patient;
an incise layer extending across each of the first, second, third, and fourth fenestrations, the incise layer having an adhesive side that is adapted to adhere to the patient; and
an absorbent reinforcement panel disposed about the first, second, third, and fourth fenestrations, the reinforcement panel being positioned on the upper surface of the base sheet and having a reinforcement fenestration that is aligned with each of the first, second, third, and fourth fenestrations.

28. A surgical drape comprising:
a base sheet having an upper surface, a lower surface, a forward edge, and a rearward edge;
a first fenestration formed in the base sheet through which a surgical procedure may be performed, the first fenestration formed in the base sheet such that when the drape is positioned on a patient, the first fenestration is disposed proximate to the radial artery in the right arm of the patient;
a second fenestration formed in the base sheet through which a surgical procedure may be performed, the second fenestration formed in the base sheet such that when the drape is positioned on a patient, the second fenestration is disposed proximate to the femoral artery in the right leg of the patient;
a third fenestration formed in the base sheet through which a surgical procedure may be performed, the third fenestration formed in the base sheet such that when the drape is positioned on the patient, the third fenestration is disposed proximate to the radial artery on the left arm of the patient;
a fourth fenestration formed in the base sheet through which a surgical procedure may be performed, the fourth fenestration formed in the base sheet such that when the drape is positioned on the patient, the fourth fenestration is disposed proximate to the femoral artery on the left leg of the patient;
an incise layer extending across each of the first, second, third, and fourth fenestrations, the incise layer having an adhesive side that is adapted to adhere to the patient; and
a slip-resistant absorbent reinforcement panel disposed about the first, second, third, and fourth fenestrations, the reinforcement panel being positioned on the upper surface of the base sheet and having a reinforcement fenestration that is aligned with each of the first, second, third, and fourth fenestrations.

29. A surgical drape comprising:
a base sheet having an upper surface, a lower surface, a forward edge, and a rearward edge;
a first fenestration formed in the base sheet through which a surgical procedure may be performed, the first fenestration formed in the base sheet such that when the drape is positioned on a patient, the first fenestration is disposed proximate to the radial artery in the right arm of the patient;
a second fenestration formed in the base sheet through which a surgical procedure may be performed, the second fenestration formed in the base sheet such that when the drape is positioned on a patient, the second fenestration is disposed proximate to the femoral artery in the right leg of the patient;
a third fenestration formed in the base sheet through which a surgical procedure may be performed, the third fenestration formed in the base sheet such that when the drape is positioned on the patient, the third fenestration is disposed proximate to the radial artery on the left arm of the patient;
a fourth fenestration formed in the base sheet through which a surgical procedure may be performed, the fourth fenestration formed in the base sheet such that when the drape is positioned on the patient, the fourth fenestration is disposed proximate to the femoral artery on the left leg of the patient;
an incise layer extending across each of the first, second, third, and fourth fenestrations, the incise layer having an adhesive side that is adapted to adhere to the patient;
an absorbent reinforcement panel disposed about the first, second, third, and fourth fenestrations, the reinforcement panel being positioned on the upper surface of the base sheet and having a reinforcement fenestration that is aligned with each of the first, second, third, and fourth fenestrations; and
a pouch attached to the drape and having a malleable strip to maintain the pouch in an open position.
